(19) United States
(12) Patent Application Publication
(10) Pub. No.: US 2009/0155354 A1
(43) Pub. Date: Jun. 18, 2009

(54) DISPENSING ENCAPSULATED LIQUIDS INTO BODY CAVITIES

(76) Inventors: Barbara Wanamaker McLean, Somis, CA (US); Sherry Thomas, North Hollywood, CA (US)

Correspondence Address: MARVIN E. JACOBS, ESQ., 1767 MARISOL DRIVE, VENTURA, CA, 93001, US

(21) Appl. No.: 11/957,032

(22) Filed: Dec. 14, 2007

(51) Int. Cl. A61K 9/48; A61P 35/00; A61P 31/00; A61P 15/18

(52) U.S. Cl. 424/452

(57) ABSTRACT

Body tissue or cavities are treated by inserting a membrane module containing a substance for treating a condition of the cavity or tissue. The substance is enclosed in a membrane that melts, dissolves, or dissipates in the environment of the cavity such as a vagina to release the substance into the cavity.

### TECHNICAL FIELD

This invention relates to a method for delivering substances into the body, and more particularly, the present invention relates to delivering the substance to a natural body cavity while encapsulated in a membrane selected to release the substance in the environment of the cavity.

### BACKGROUND OF THE INVENTION

The principal clinical methods of administering substances to the body are oral administration of liquids or solids in pill or powder form or encapsulated solids. Liquids can be administered by injection. In some instances liquids or solids are administered to body cavities such as the ears, nose, eye sockets, mouth, rectum, or vagina. In some instances substances such as liquids, gels, creams, microspheres, suppositories, powders, or radioactive seeds are surgically implanted directly in internal organs. The current methods for delivery of substances into the body either manually or surgically, are often ineffective. They usually require careful measuring of dosage with each administration and often there is a loss of the substance during administration that results in inadequate dosing. Certain agents need to be precisely and quantitatively measured. Actual delivery is often cumbersome, inaccurate, and messy. Delivery into certain cavities is difficult and/or often impossible to administer accurately.

Current delivery systems for administering substances into body cavities such as suppositories which are usually inserted into the rectum or vagina cannot deliver a liquid, cream or gel effectively or at a correct volume. They may be slow to dissolve and are easily expelled by contractions or gravity before the full amount of the substance to be delivered is administered. Since it is impossible to determine how much of the substance was absorbed, another administration may result in overdosing. Because suppositories are in a partially solid or completely solid form, the dissolution time may be prolonged or not occur at all.

Hard tablets can irritate tissue, can easily be expelled, dissolve slowly before adequate absorption, or cause inconsistent coating of tissue in the area to be treated.

Creams, gels and lotions are not only messy and cumbersome to administer, but often a significant portion of the dosage is lost and wasted on the delivery instrument and/or outside the cavity.

It is difficult to administer an accurate dosage of liquid into some body cavities and tissues. As with creams and gels, they are often messy and cumbersome to administer, and often a significant portion of the dosage is lost and wasted by being expelled from the cavity during administration or absorption, and is retained on or in the administration instrument.

Current products on the market often are difficult to dispense evenly and/or deeply. Dryness and other related symptoms, which include but are not limited to irritation, rawness, infection of body cavities is a serious, often painful, condition in many individuals. This dryness results
in body tissue becoming more susceptible to infection, irritation and bleeding due to miniscule cracks harboring bacteria or viruses;
painful insertion of medications and instruments, both medical and non-medical;
painful or impossible sexual relations;
itching, irritation, and/or discomfort;
urinary urgency, frequency spasms, or pain
rectal irritation or spasms.

### Causes of Dryness:

Body cavity dryness can occur due to several factors:
The natural aging body hormones balance changes;
Nephrectomy hysterectomy, removal of the ovaries, or other surgeries creating the body's hormones to be depleted;
Medical therapy, such as radiation or chemo therapy, which create an imbalance in body chemistry;
Decrease in hormones due to such factors as premature ovarian failure, hypothyroid, or Sogreins syndrome;
Hereditary, genetic, or medical predisposition;
Depression or other psychological conditions;
Side effect of many medications;
Certain cavities are naturally non-lubricated, such as the rectum or mouth.

Current Products on the Market Include:
various consistencies of product contained in a tube or bottle, in the form of a gel or viscous liquid such as KY Liquid Lube, Vagisil Liquid, KY Moisture Enhancing Liquid, Astroglide, and many under store labels. These products are messy to apply to the instrument to be inserted, which leaves the cavity dry. They are messy and difficult to apply or insert inside the body cavity, especially to any depth or specific location. Their application is obvious and usually awkward.
Replens Vaginal Moisturizer, a tube with a plastic applicator. One fills the applicator from the tube, inserting the rigid applicator into the body cavity to dispense moisture. The applicator must be reused, which can create hygienic concerns if it is not properly cleaned after each use. The rigid applicator can be painful, too large, or cause irritation and trauma on insertion.
Rephresh, a small amount of lubricant in a plastic applicator, which is inserted into the body cavity and squeezed to release contents. This product is expensive, approximately $2.50 per application, with 8 to a box. Currently it is not widely available but can be found in some pharmacies. The applicators are plastic, therefore may be uncomfortable when administering into a dry cavity.
Vagifern or Gynelotrirnin tablets are often difficult to insert causing tissue trauma and pain. They are easily expelled and sometimes not retained inside the cavity.
Various sized bottles of viscous liquid which is to be applied by hand either to the instrument to be inserted and/or to the inside of the cavity. Like the tube products above, these are difficult to administer effectively and messy.

There is a need for a product which can be effectively inserted into, lubricate, and treat body cavities conveniently, neatly, discretely, and efficiently at an affordable price. The cavity lubricant or treatment should be administered without disruptive fuss, without messy handling, and without awkward trial and error. The release of the substance should not occur until the module is located in the target body cavity or tissue.

Recently (April 2007) a product (LIQUIBEADS™ OVULES®) became available for delivering lubricants and/or antibacterial or antifungal agents to a body cavity, such as a vagina or an anus. An OVULE® contains a dispersion of LIQUIBEADS™ which are believed to be similar to KY Brand of vaginal gel lubricant. The ingredients stated on the package insert are dimethicone, gelatin, glycerin and dimethiconol. The Ovule is placed in one end of a plastic applicator. The applicator is inserted in the vagina and a plunger is pushed to place the OVULE insert in the vagina. The OVULE releases the LIQUIBEADS™ which coat onto the walls of the vagina. The applicator and plunger are removed and discarded.

At least one half hour is required to coat the lubricant onto the walls of the vagina and complete coating of the walls is facilitated by exercise before intercourse. The treatment is effective for at least several days.

U.S. patent application Ser. No. 0070065504 discloses a solid dosage form of an Ovule containing an effective amount of a single dosage form of a treatment substance such as an antibiotic, an antifungal, an antiviral, etc. The solid ovule coating must melt in the body cavity and slowly spreads through the vaginal cavity. Again, exercise can speed up the melting and spread of the solid. For example a dose of solid fat such as hydrogenated vegetable fat and an active ingredient can be encased in a soft gelatin capsule. The capsule film can be formed of gelatin and glycerin.

Common to the use of the OVULES is the administration of LIQUIBEADS™ which require at least one-half hour to distribute in the body cavity and are effective for several days to treat an infected condition in the cavity. Also, the use of an applicator is preferred so that the capsule shell and LIQUIBEADS™ are not exposed to the environment of the cavity during insertion.

### STATEMENT OF THE INVENTION

The invention relates to the administration of non-solid substances of various consistencies into a body cavity. The substances are enclosed in a membrane vehicle which will melt, dissolve or dissipate to release the substance when placed in the physical environment of the cavity such as melting at the temperature of the cavity or the pH or other condition of the cavity causes the membrane vehicle to dissolve or disintegrate.
1) The vehicle can consist of a thin film of membrane material.
2) The membrane will melt, dissolve, or dissipate in an acceptable temperature range, thereby responding to internal body heat or pH level to release its contents.

With the delivery system of the invention it is preferred that the membrane capsule be inserted into the cavity rather than the tissue of the cavity being in contact with the plastic barrel of the applicator as used with OVULES. The membrane melts, dissolves or dissipates and quickly coats the walls with treatment substance as it is released into the cavity. The membrane dissipates quickly in the environment of the body cavity and the low viscosity liquid is released and disperses evenly, deeply and quickly to cover all internal tissue. Manual insertion is more comfortable than use of an applicator which can hurt sensitive tissue on insertion and withdrawal of the applicator. Also the OVULE requires waiting at least 30 minutes after insertion to allow the encasement to react before lubrication can be initiated.

In the delivery module of the invention, the thickness and composition of the membrane film are adequate to maintain shape, form and integrity of the module capsule as it is handled and manually inserted into a cavity.

In the present delivery system the membrane encasing the substance can be any solid film that is impervious to the encased liquid, is stable and has long shelf life in a storage environment. The membrane retains its integrity while being stored and handled and can be inserted deeply into a body cavity before releasing the liquid. The membrane can be formed of synthetic polymers that are pharmacologically acceptable such as solid polymers of polyakylene oxides such as polyethylene oxide. Preferred membrane films are formed of natural materials suitably hard vegetable waxes such as coconut oil esters, gelatin, pectin, gums such as guar gum. A natural film can be formed from an ester of vegetable or nut oil containing from 20-40 carbon atoms such as a coconut oil ester. The oil may be hydrogenated to increase its hardness and melting point.

These and other features and attendant advantages of the invention will become apparent as the description proceeds.

### BRIEF DESCRIPTION OF THE DRAWINGS

FIG. 1 is a front view in elevation of a first embodiment of a capsule for delivering a liquid into a body cavity;

FIG. 2 is a front view in elevation of a second embodiment of capsule suitable for digital insertion;

FIG. 2A is a sectional view taken along line 2A of FIG. 2;

FIG. 3 is a view in section of a capsule within a capsule embodiment;

FIG. 4 is a schematic view of a capsule divided into two compartments;

FIG. 5 is a schematic view of an alternative version of a two compartment capsule;

FIG. 6 is a schematic view of a membrane encapsulated substance or microspheres suspension; and

FIG. 7 is a schematic view of a capsule divided into four compartments by membrane walls.

### DESCRIPTION OF THE INVENTION

The capsule utilized in the invention can be liquid or gel encapsulated in a membrane that melts, dissolves or dissipates in the body cavity. The encapsulated liquid or gel can contain a unit dosage amount of a medicine suitable to treat and cure the infected or other condition of the target tissue.

The outer membrane can take various shapes and/or sizes and or thicknesses, melting temperatures or chemical compositions to accommodate the specific need of a substance to be delivered or to accommodate the specific body cavity proportions or to control timing of administration of the encapsulated liquid, gel or suspension.

The membrane may be layered to contain more than one substance for delivery. Outer membrane will melt, dissolve or dissipate to release the first substance, and then the next membrane will melt to release a subsequent substance. This second substance or dosage may be a suspension within the encapsulated liquid or may be a part of a partitioned module.

Various thicknesses, melting temperatures, or chemical compositions of the membrane may be used to vary timing of administration of the contents of the capsule.

The specific chemistry of the membrane may have to be altered or coated with a protective outside layer to prevent premature reactions with substances present in the cavity or entrance to the cavity.

Capsules may be produced with contents targeting other problems including, but not limited to:
a) Cavity pH balance;
b) Candida control;
c) Birth control;
d) Tissue health;
e) Hormone balance;
f) Treatment of sexually transmitted diseases;
g) Lactobacillus homeo stasis;
h) Treatment of cancerous target tissue
i) Treatment of other medical conditions; and
j) Treatment of bacterial or viral infection

The membrane capsule can be produced empty. The physician or pharmacist may inject a therapeutic liquid, gel, powder or other substance into the capsule.

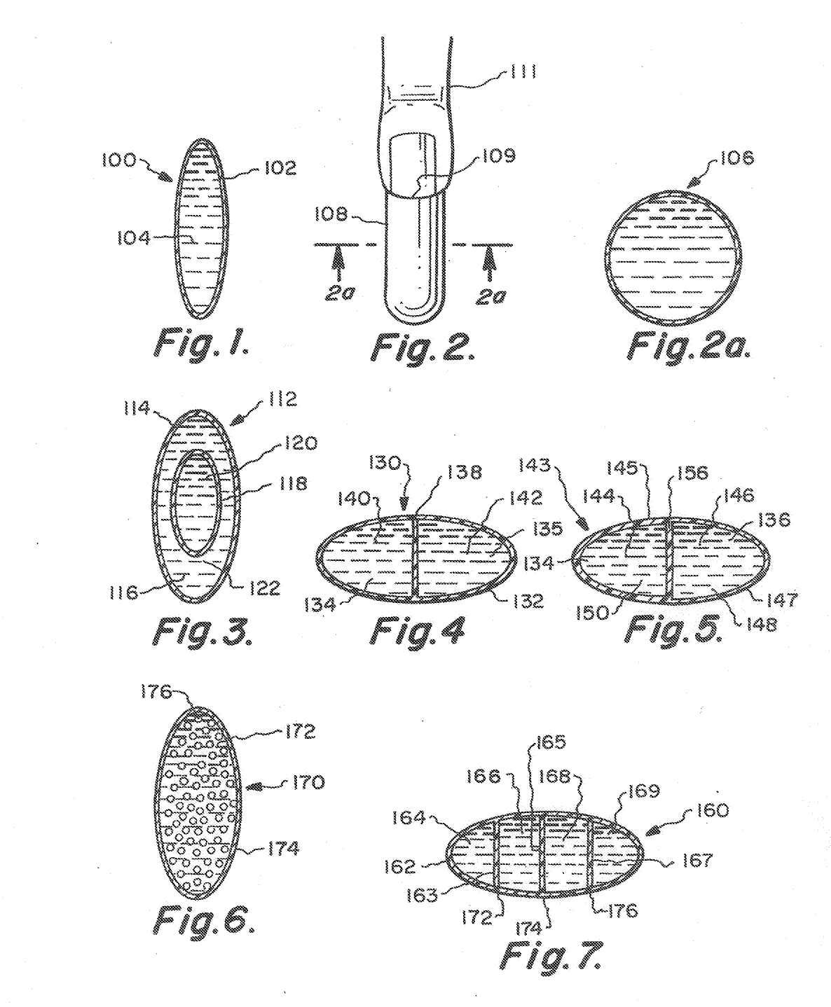

Referring now to FIG. 1, a capsule 100 can take the form of an elongated, thin, oval membrane 102 encapsulating a body 104 of liquid. FIGS. 2 and 2a illustrate a wide capsule 106 having a convex distal end 108 and a concave proximal end 109 in which the top of a finger 111 can be placed during insertion of the capsule 106. The distal end 108 can be convexly shaped or pointed to aid in insertion.

FIG. 3 illustrates a capsule 112 having an outer membrane skin 114 which encapsulates a first body of liquid 116 and an inner membrane and film 118 which encapsulates a second body of liquid 120. The liquids 116, 120 can be the same and delivery will occur in stages allowing a push rod as described in my co pending application entitled “APPARATUS AND METHOD FOR DELIVERY OF SUBSTANCES INTO TARGET TISSUE”, the disclosure of which is incorporated herein by reference to breach the outer membrane skin 114 to expel the first body of liquid 116 into tissue or the liquid can be released as the membrane dissipates, dissolves or melts by other process. After absorption a body of liquid 116, a push rod is again pressed to rupture the inner capsule 118 which then expels the second body of liquid 120. The liquids 116 and 120 can contain different agents released in tandem as membrane dissipates by melting or dissolving such as liquid 116 containing blood flow and liquid 120 containing a different therapeutic substance.

FIG. 4 illustrates a two compartment capsule 130 in which the interior of a suitably oval-shaped membrane 132 is partitioned into two chambers 134 and 135 by wall 138. The liquids 140, 142 contained within chambers 134 and 136 will be dispensed simultaneously on breach of the outer membrane film 132 or in sequence on breach of chambers 134, 135 sequentially. A push rod can be advanced stepwise to sequentially disrupt the membranes enclosing chambers 134, 135 and sequentially release the bodies of liquid 140, 142. In case of thermally melt able membrane the liquids release when the membrane melts.

In FIG. 5 the first chamber 134 in the capsule 143 has a thicker membrane 145 than the membrane 147 surrounding the second chamber 136. The bodies of liquids 144, 146 are contained within compartments 148, 150. However, the thickness of the outer membrane 145 connected to a partition wall 156 is thicker than the other portion the membrane 147 also connected to the partition wall 156. The thinner membrane 147 will be breached first and deliver the contained liquid 146 to targeted tissue. The thicker membrane 145 will breach or melt, dissipate or dissolve at a later time.

FIG. 6 shows a capsule 170 in which the liquid 172 encapsulated by the membrane 174 contains a dispersion of solid particles 176.

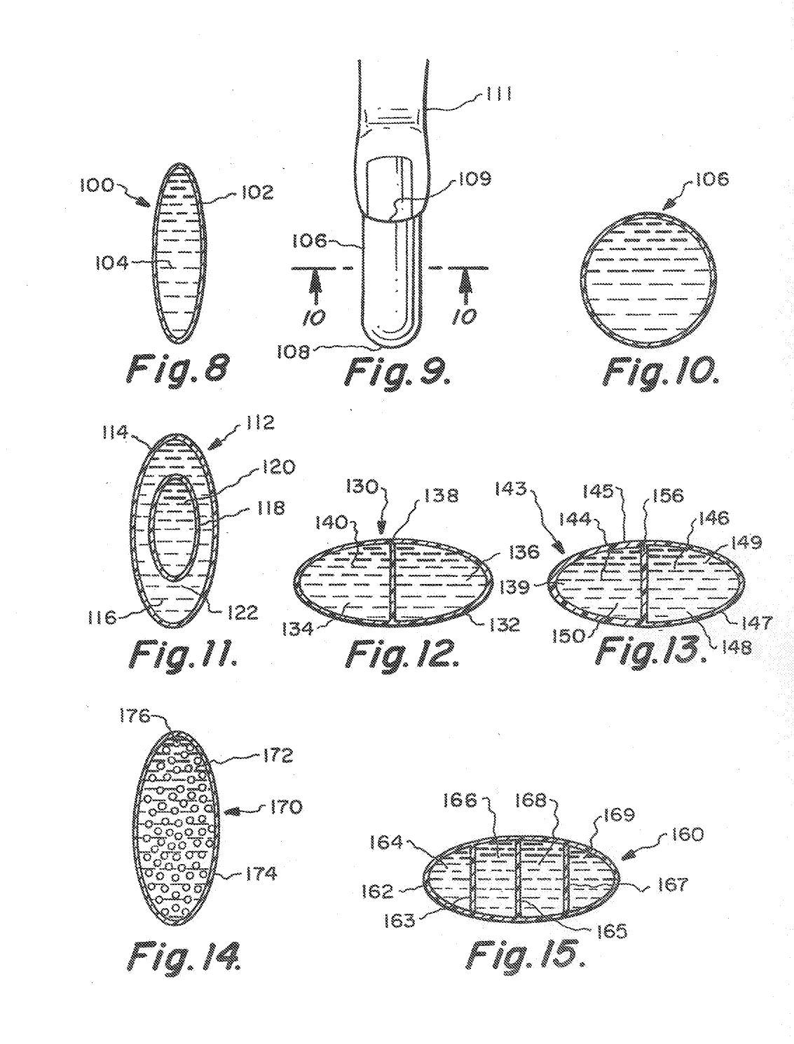

In FIG. 7 a capsule 160 having an outer membrane 162 is partitioned into 4 chambers 164, 166, 168, 169 by means of walls 172, 174, 176 which extend across the membrane 162.

It is to be realized that the preceding description is for the purpose of enabling one to understand the invention and is not intended to limit the invention except as defined in the following claims.

1. A capsule comprising a thin outer membrane surrounding a body of liquid or gel for treatment within tissue or a body cavity. Said membrane being impervious to said substance, liquid or gel under ambient conditions external to said cavity but becoming liquid permeable when within said cavity to dispense said liquid onto walls of tissue surrounding said cavity.

2. Various thicknesses, melting temperatures, or chemical compositions of the membrane may be used to vary timing of administration of the contents of the capsule.

3. A capsule according to claim #1 which the period to dispense the liquid or gel is controlled by the thickness, melting temperature or chemical composition of the membrane.

4. A capsule according to claim #1 in which liquid or gel is selected from the group consisting of creams or ointment.

5. A capsule according to claim 1 in which the tissue within the body cavity is dry and the liquid or gel comprises a liquid, gel or cream for treating said dryness.

6. A capsule according to claim 2 in which the cavity is a vagina and the liquid or gel is a lubricant for vaginal tissue.

7. A capsule according to claim 1 in which the membrane melts within the body cavity to dispense the liquid or gel.

8. A capsule according to claim 1 in which the membrane remains solid when the capsule is stored at a temperature no more than 80° F.

9. A capsule according to claim 1 in which the capsule is divided into a plurality of compartments.

10. A capsule according to claim 6 in which the membrane enclosing a first compartment dispenses the substance contained in the first compartment before the membrane enclosing a second compartment dispenses the liquid contained within the second compartment.

11. A capsule according to claim 7 in which the thickness of the membrane enclosing the first compartment is or may be greater than the thickness of the membrane enclosing the second compartment, to vary or prolong the timing that the second module melts, dissolves or dissipates.

12. A capsule according to claim 8 in which the membranes melt, dissolve or dissipate in the environment of the cavity.

13. A capsule according to claim 1 in which the tissue in the cavity before insertion of the capsule is suffering from a clinical condition.

14. A capsule according to claim 10 in which the condition is selected from the group consisting of:
pH balance;
Candida;
Birth control;
Sexually transmitted disease;
Lactobacillus homeo stasis
Cancer; or
Bacterial or viral infection

15. A method of treating an animal having a body cavity requiring treatment for a condition comprising the steps of inserting into the cavity a body of liquid, gel, cream, powder, ointment or microspheres containing a substance for treatment of said condition, said body being enclosed in a membrane; dispensing the body of liquid from the membrane enclosure while the membrane enclosure is in said cavity; and/or coating walls of the cavity with said body containing the substance.

16. A method according to claim 12 in which the cavity is a vagina, the condition is dry vaginal tissue; and the substance is vaginal lubricant.

17. A method according to claim 12 in which the liquid is dispensed by melting the membrane within the cavity to release the liquid.

18. A method according to claim 1 in which the liquid or gel is dispersed by mechanically breaching the membrane.
